(19) United States
(12) Patent Application Publication
(10) Pub. No.: US 2012/0197317 A1
(43) Pub. Date: Aug. 2, 2012

(54) QUICK RELEASE SPINAL IMPLANT INSERTION DEVICE

(76) Inventors: Alejandro Lezama, Milwaukee, WI (US); Jennifer M. Schneider, Germantown, WI (US)

(21) Appl. No.: 13/362,420

(22) Filed: Jan. 31, 2012

Related U.S. Application Data
(60) Provisional application No. 61/438,425, filed on Feb. 1, 2011.

(51) Int. Cl. A61B 17/88

(52) U.S. Cl. 606/86 A

(57) ABSTRACT

This invention is a novel, quick release spinal implant inserter, insertion device or holder/dispenser. It has the advantage of providing enhanced surgeon control of the prosthesis insertion process including the ability to insert and manipulate intervertebral spinal fusion implants or devices of many different sizes.

### BACKGROUND OF THE INVENTION

Intervertebral discs, located between the endplates of adjacent vertebrae, stabilize the spine, distribute forces between vertebrae and cushion vertebral bodies. A normal intervertebral disc includes a semi-gelatinous component, the nucleus pulposus, which is surrounded and confined by an outer, fibrous ring called the annulus fibrosis. In a healthy, undamaged spine, the annulus fibrosis prevents the nucleus pulposus from protruding outside the disc space.

Spinal discs may be displaced or damaged due to trauma, disease or aging. Disruption of the annulus fibrosis allows the nucleus pulposus to protrude into the vertebral canal, a condition commonly referred to as a herniated or ruptured disc. The extruded nucleus pulposus may press on the spinal nerve, which may result in nerve damage, pain, numbness, muscle weakness and paralysis. Intervertebral discs may also deteriorate due to the normal aging process or disease. As a disc dehydrates, the disc space height will be reduced leading to instability of the spine, decreased mobility and pain.

Sometimes the only relief from the symptoms of these conditions is a discectomy, or surgical removal of a portion or all of an intervertebral disc followed by fusion of the adjacent vertebrae. The removal of the damaged or unhealthy disc can permit the disc space to collapse. Collapse of the disc space can cause instability of the spine, abnormal joint mechanics, and premature development of arthritis or nerve damage, in addition to severe pain.

Bone grafts are sometimes used to fill the intervertebral space to prevent disc space collapse and promote fusion of the adjacent vertebrae across the disc space. For example, in the Smith-Robinson technique for cervical fusion, the surgeon prepares the endplates of the adjacent vertebral bodies to accept a graft after the disc has been removed. The endplates are generally prepared to be parallel surfaces with a high speed burr. The surgeon sculpts the graft to fit tightly between the bone surfaces so that the graft is held by compression between the vertebral bodies. The bone graft is intended to provide structural support and promote bone ingrowth to achieve a solid fusion of the affected joint.

Unfortunately, the use of bone grafts presents several disadvantages. Autografts, bone material surgically removed from the patient, can be undesirable because they may not yield a sufficient quantity of graft material. The additional surgery to extract the autograft also increases the risk of infection and blood loss. The structural integrity at the donor site can be reduced. Furthermore, some patients complain that the graft harvesting surgery is more painful than the fusion surgery.

Prosthetic implants can be used to prevent collapse of the disc space. The implant must provide temporary support and allow bone ingrowth. Success of the discectomy and fusion procedure requires the development of a contiguous growth of bone to create a solid mass.

Inserters or implant holders are instruments used to insert, and often to manipulate, a spinal implant during spinal intervertebral body fusion surgeries. Many inserters utilize prongs or threads to stabilize the implant during the surgical procedure. See, e.g., U.S. Patent Application Publication 2004/0267275 (“Cournoyer et al.”) (WO 2008/146983 (“Lee et al.”), the teachings of which are incorporated by reference herein. The implant insertion device disclosure of U.S. Pat. No. 6,066,174 to Farris i.e., FIGS. 7-13 and related descriptive material also is incorporated by reference herein. These conventional inserters suffer the drawback that holding structures may become disengaged during insertion or manipulation of the implant. The stability achieved by these features is balanced by the size of the required holding feature on the implant, and the ability to maintain a holder-implant interface with a slim profile to allow complete insertion of the implant.

This invention is a novel, quick release spinal implant inserter, insertion device or holder/dispenser. It has the advantage of providing enhanced surgeon control of the prosthesis insertion process including the ability to insert and manipulate intervertebral spinal fusion implants or devices of many different sizes.

### A. BRIEF STATEMENT OF THE INVENTION

In accordance with one aspect of the invention, implant insertion devices or inserters are provided for inserting implants such as intervertebral spacers. A device according to this invention include: a handle, a shaft having a proximal end attached to the handle and a distal end and a pair of jaws each having a first end attached to the distal end of the shaft, a free second end and a gripping element at the second end. The free ends of the jaws are movable between gripping positions to grasp the implant between the gripping elements and releasing positions to release the implant. The first ends of the jaws form a first angle in the releasing positions and a smaller second angle in the gripping positions. The jaws can be biased apart to or toward the releasing positions. A device according to this invention also optionally includes a threaded lock mechanism which permits fixing or locking a given jaw position so as to permit, for example, manipulation of the intervertebral implant, e.g., repositioning during the implant procedure.

The devices of this invention also include forcing means for forcing the jaws together towards the gripping positions. The jaws have a bias toward being open or disengagement created in their manufacture. In one aspect, the forcing means can includes a hollow sleeve slideably mounted on the shaft and defining an inner bore for receiving the shaft and the first or proximal ends of said jaws. The sleeve is slideable between a first position adjacent the handle (i.e., a more proximal position) and a second engaging position (i.e., a more distal position) spaced from the first position in a location between the first position and the free ends of the jaws. The sleeve forces the jaws together when the sleeve is in the engaging, more distal position to engage or “lock on” to an implant.

It is noted that the “jaws” or implant engaging portions thereof include an inwardly-directed, distal prong or hook which engages and holds an implant when the holder is in its engaging position.

A particular preferred sleeve of this invention has a partially circumferential, proximally stepped distal end configuration which provides plateaus, facets or steps with differing radial separation. The plateaus or steps mate with cooperating surfaces on intervertebral prostheses of differing vertical dimension or vertebral height and different geometries (i.e., the inserts are of different sizes and configurations) permitting a single inserter of this invention to be used with variously sized implants. This invention also utilizes a quick release holding feature, or structure which incorporates prongs and a preferred unique sleeve geometry. Together, these features provide stability to the implant-holder assembly, while maintaining a very slim profile.

In some embodiments, a device of this invention includes a locator pin means extending distally from the shaft. The locator pin cooperates with, e.g., a hole, on an implant to more securely hold it.

The device has an optional threaded lock means or mechanism, e.g., a threaded collar and a cooperating threaded lock nut that may be engaged if the surgeon desires increased control to manipulate the implant during or after placement.

Additionally, the instrument may be easily disassembled to assure proper cleaning, which can be a challenge with elaborate implant holder designs. Cleaning is important for operation of the device to prevent failures and to prevent infection.

This invention is an elegant spinal implant holder/dispenser that is straightforward and easy to use, yet accommodates a broad range of implant sizes and implant configurations. Providing the surgeon with a stable implant-inserter assembly significantly enhances the likelihood of success of the surgery.

Typically, medical device companies which market spinal interbody fusion devices provide the tools required to insert the product along with the implants themselves. The invention can be part of the instrument kit or set provided to the surgeon to aide in the insertion of interbody implants. It will be appreciated that since a device of the invention can insert many sizes of fusion devices that substantial reductions of inventory, shipping, and storage casts are obtained.

Prior art includes threaded holding features, prongs, or a combination thereof. Threads can be time consuming to assemble/disassemble in the operating arena, and may become unstable if not threaded completely. Manipulation in a direction opposite the thread may cause the inserter to become unstable. Prongs provide some medial-lateral stability; however the size of the prongs may limit the compatibility with smaller devices, or if small enough to accommodate these sizes, may not provide sufficient stability to manipulate the implant intraoperatively. When designed large enough to provide stability, both threads and prongs can limit the design of the implant geometry.

Intervertebral body fusion devices are typically packed with bone graft material to facilitate fusion. The size of the open pore within the cage is desired to be as large as possible for both graft packing, and with radiopaque materials such as metal cages, for radiographic visualization of the fusion postoperatively.

The invention utilizes a quick release holding feature, which incorporate prongs and a novel distal end sleeve geometry. The sleeve geometry permits a single device of this invention to be used to implant intervertebral spinal fusion devices of various sizes, thereby reducing inventory, equipment and clean-up and storage costs. Together, these features, their combination and sub-combinations, provide stability to the implant-holder assembly, while maintaining a very slim profile. The device or tool also reduces inventory and surgical time by allowing use of single instrument to fully insert and manipulate cage. (Secondary instrument-tamp typically used).

### BRIEF DESCRIPTION OF THE DRAWINGS

The spinal implant holder of this invention is illustrated in the following figures which are intended to be illustrative and not limiting:

FIG. 1 is a side perspective view of an inserter of this invention.

FIG. 2 is a section view of the inserter of FIG. 1 taken along line 2-2 of FIG. 1 with the following structure designations:

| ITEM NO. | DESCRIPTION |
|---|---|
| 1 | HANDLE |
| 2 | COLLAR |
| 3 | CAP |
| 4 | SPRING |
| 5 | PIN |
| 6 | SHAFT |
| 7 | LOCK NUT |
| 8 | SLEEVE |

FIG. 3 is an enlarged partial section view of the distal end of the inserter FIG. 1 with parts broken away and others shown in section at circle “M” in FIG. 2, showing the novel distal end configuration of a device of this invention.

FIG. 4 is an enlarged sectional view of the handle and proximal portion of the shaft of an inserter of FIG. 1.

FIG. 5 is a side perspective view of the sectioned inserter of FIG. 4.

FIG. 6 is an exploded view of an inserter of FIGS. 1-5. Note that collar 2 could be integral with sleeve 8 and be a portion of the proximal end thereof.

FIG. 7 is a perspective view of an inserter of this invention with an intervertebral implant held at its teeth.

FIG. 8 is a perspective view of a system of this invention comprising inserter of this invention as it is engaging an intervertebral insert or implant 100.

FIGS. 9A-9D are perspective views of an inserter of this invention with several sizes of implants.

### DETAILED DESCRIPTION OF THE INVENTION

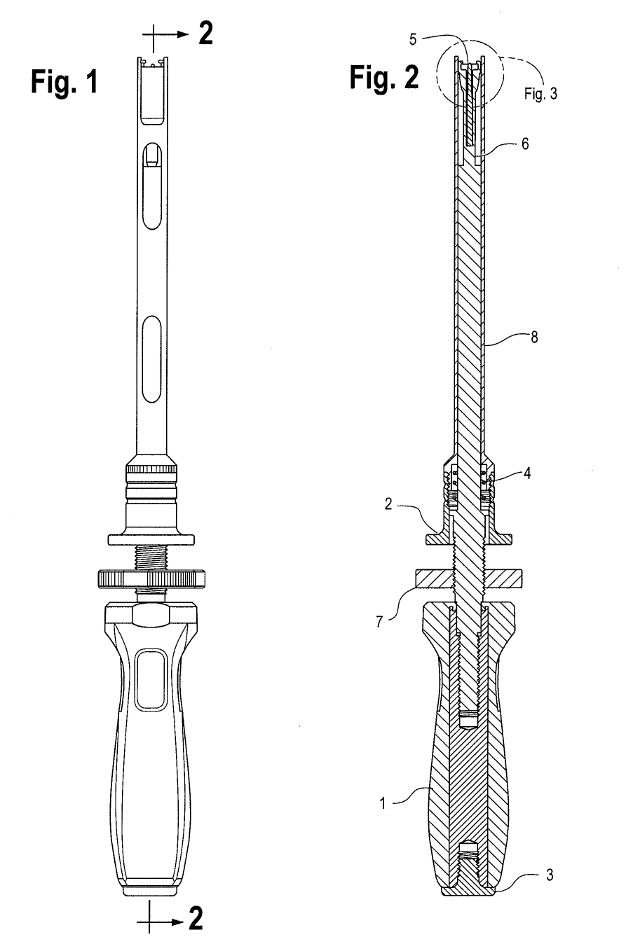

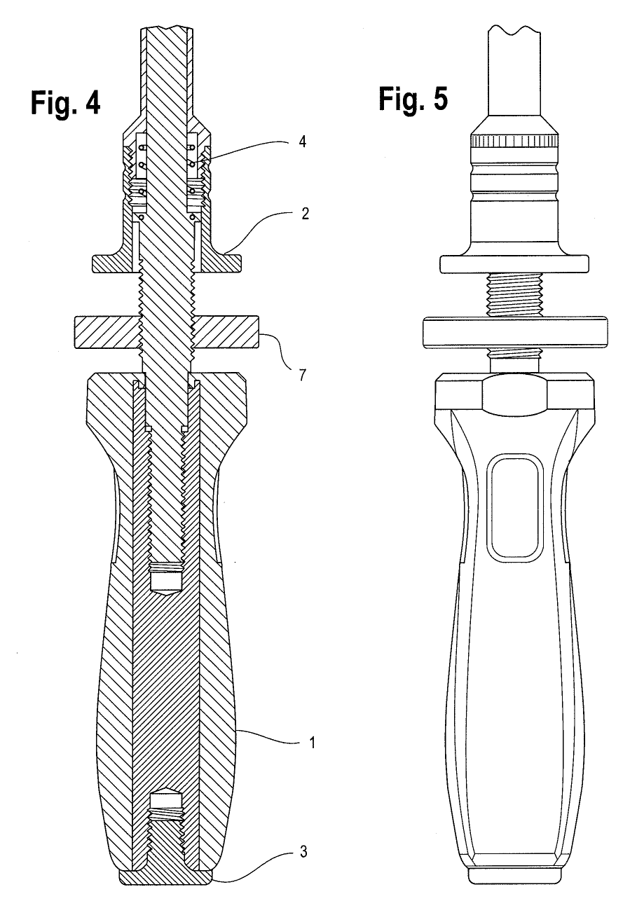

For the purposes of promoting an understanding of the principles of the invention, reference will now be made to the embodiments illustrated in the drawings (FIGS. 1-9D). It will nevertheless be understood that no limitation of the scope of the invention is thereby intended, such alterations and further modifications in the illustrated devices, and such further applications of the principles of the invention as illustrated therein being contemplated as would normally occur to one skilled in the art to which the invention relates.

The implant holder of this invention is a simple but elegant holder/implant-direction device that includes a spring-loaded mechanism, which urges the collar distally to close the prongs, which capture the implant when closed and a central locating pin that allows the medical professional to guide the implant itself onto the holder. The holder can be disassembled for ease of cleaning and quickly reassembled for use. The holder utilizes a very simple quick release of the implant which can be accomplished using the fingers of one hand to pull the collar proximally. Pulling the collar/sleeve proximally (The frame of reference regarding the indication of proximal and distal is from that of a medical professional using the device.) against the resistance provided by the spring permits the prongs to spread and to disengage the openings in the sides of the implant. Release of the collar and concomitant distal movement of the sleeve in response to the biasing force of the spring engages the implant and positions it correctly for use during surgery. This permits the implant to be engaged with one hand and to be disengaged with one hand.

For additional stability the surgeon may use the lock mechanism which can be threaded distally to face the proximal end of the sleeve. Engagement between the lock mechanism or locknut and the proximal end of the sleeve adjacent the collar essentially “fixes” the implant to the implant holder itself. This prevents the collar from moving proximally and potentially releasing the implant prematurely i.e., before release is needed, during the surgical procedure. Having locked the implant onto the implant holder by means of the locknut butting against the collar, the implant itself may then be manipulated by the medical professional in the surgical process using its handle.

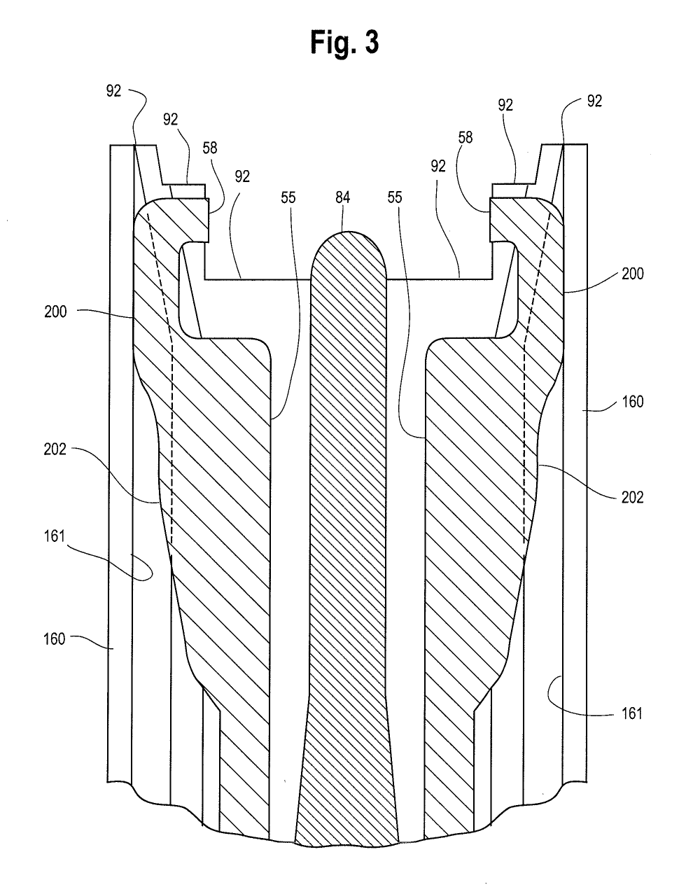

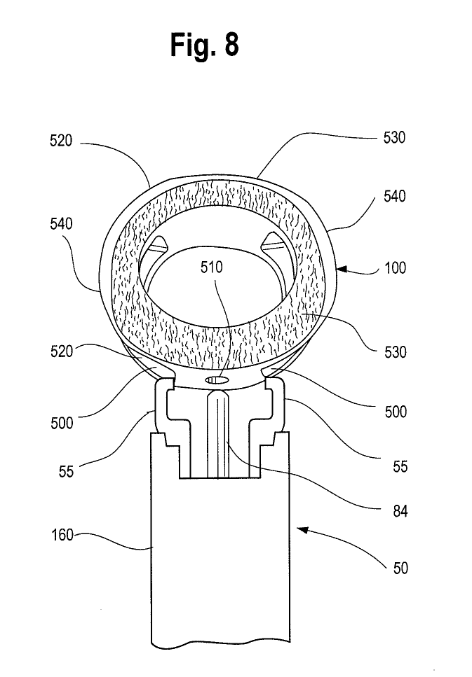

It is to be noted (FIG. 3, discussed below) that the extreme distal end of the holder sleeve or gripping element has a series of facets or surfaces which are disposed perpendicular to the axis of the holder itself and proximally stepwise and engage the sides of an implant. The facets provide the accommodation needed if implants from small to very large are to be implanted using this device. Thus, in a very real sense the device is one-handed in use, lockable, implant retention of implants having a large range of different sizes and geometries. As is shown in FIG. 8, the sidewall of an implant provides the surfaces which cooperate with a tool of this invention. If the cooperative surfaces are present in the implant the rest of its configuration can be determined by medical exigencies.

It is further to be noted that both one hand engagement and one hand release of the implant are permitted in use of this holder. Its simplicity, reliability, ease of use, ease of cleaning, is provided by the combination of features of this invention including their relevant sub-combinations.

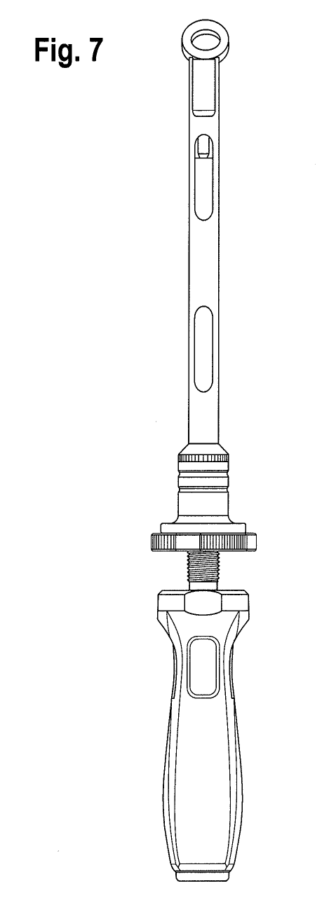

It is also to be noted that the inner shaft at its extreme distal end has the prongs machined therein. The outer surface of the prongs at the distal end of the inner shaft radiate away from the axis of the holder. This provides a surface upon which the sleeve can interact with the prongs and move the hook portion of the prongs toward the axis of the device and thus “hook” into an implant. The details of the implant engagement portion of the prongs are best seen in FIG. 3 and in FIG. 7.

Incorporated by reference herein are “Spinal Fusion Surgery” and “Anterior Cervical Spinal Fusion Surgery” both published by Spine-health. Described are surgical procedures in which the present invention could be used.

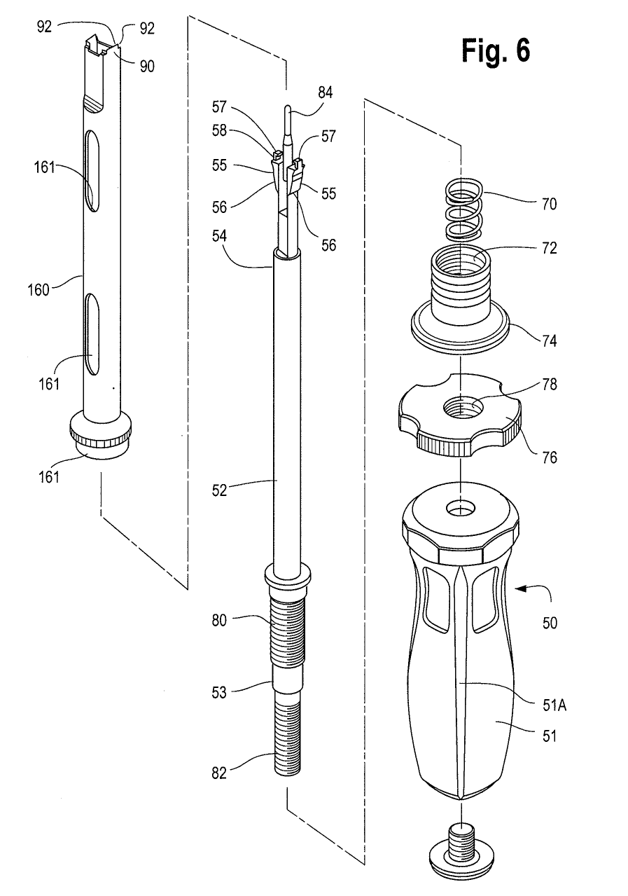

The present invention, in one aspect, is an inserter 50 as depicted in FIG. 6 for facilitating the implantation of spacers. The inserter 50 includes a handle 51a shaft 52 having a proximal end 53 extending from the handle 51 and a distal end 54 with a pair of jaws 55 each attached to the distal end 54 of the shaft 52. The handle 51 is preferably provided with knurlings, silicone 51a or other suitable patterns to enhance manual gripping of the handle. Silicone is a preferred handle material.

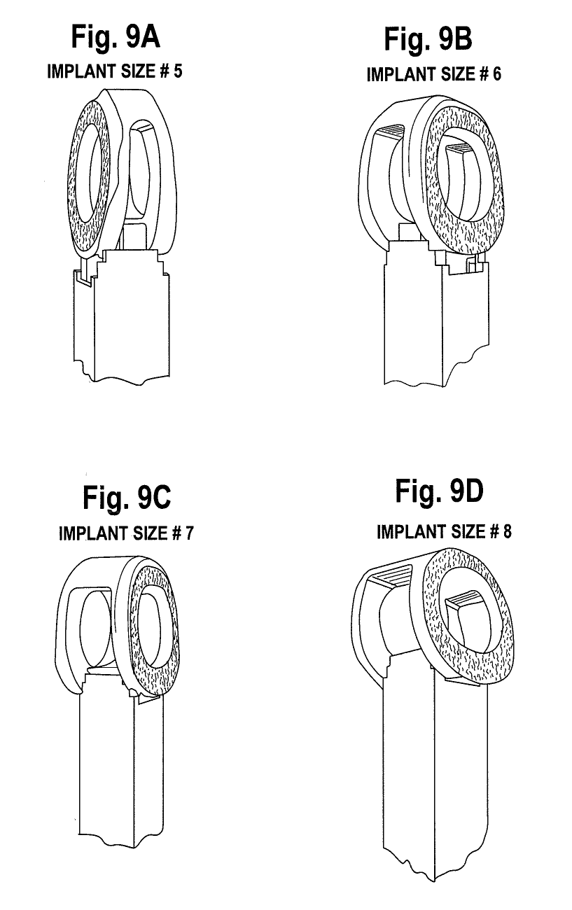

Each of the jaws 55 has a first or proximal end 56 adjacent to the distal end 54 of the shaft 52 and a free or distal second end 57. Each of the free ends 57 includes a gripping element such as a prong or hook 58 (best shown in section in FIG. 3) for engaging and gripping an implant. The free ends 57 of the jaws 55 are movable between gripping positions (FIGS. 8 and 9) to grasp the implant between the gripping elements 58 and releasing positions (FIG. 6) to release an implant 100 (FIG. 9A). Jaws 55 are biased apart to the releasing positions due to their construction.

The inserter 50 further includes an actuator or sleeve 160 operable on the jaws 55 to force the jaws 55 together. In one embodiment, the actuator is a sleeve 160 that is concentrically disposed around and slideably mounted on the shaft 52. Preferably the sleeve 160 defines an inner bore 161 for receiving the shaft 52 and the first ends 56 of the jaws 55. The sleeve 160 is slideable between a first or proximal position adjacent the handle 51 and a second or distal engaging position spaced from the first position in a location between the first position and the free ends 57 of the jaws 55.

In the use of the inserter 50, the sleeve 160 is slid along the shaft 52, and more particularly along the opened jaws 55, to push the jaws 55 together. As the jaws 55 are pushed together, the gripping surfaces 59 engage and firmly grip an implant 100 as described above. Advantageously this inserter can then be extended percutaneously or otherwise into the surgical site to implant a spacer 100 in the intradiscal space. Once the spacer is properly positioned, the sleeve 160 can be moved proximally i.e., towards the handle 51 so that the two jaws 55 spread apart to the releasing positions, thereby releasing the spacer 100. The jaws 55 can spread apart due to their natural resilience or due to another biasing means as discussed above.

An inserter of this invention also includes a biasing means such as spring 70 which urges sleeve 160 distally to close jaws 55. Spring 70 nests within internally threaded (at 72) collar 74. Collar 74 is threadingly engaged around shaft 52 at threads 80. Lock nut 76 (which is threaded at 78 also is threadingly engaged to shaft 52 via threads 82. Lock nut 76 and collar 74 engage, via their respective internal threads 72, 78 with shaft 52 threads 80, 82 to lock collar 74 in position which in turn fixes jaws 54 to permit an intervertebral prosthetic device 100 to be fixed to the inserter 50 to permit its manipulation during the insertion process. Fixing of an insert 100 to inserter 50 is assisted by distal pin 84 which cooperates with a hole in the insert.

A significant further feature of the present invention is shown in FIG. 6 (and FIG. 3) at the distal end 90 of sleeve 160. The distal end 90 of sleeve 160 is configured with facets 92 (see also FIG. 3) which proceed proximally and radially in stepwise fashion. Facets or steps 92 permit inserts 50 to be used with spinal inserts 100 of ever larger vertical height (See FIGS. 9A-9D).

Further referencing FIG. 3, there is shown the surface interaction (at 200) between sleeve 160 and the outside distal surface of jaws 55. Proximal to surface 200 are diminishing radius ramps 202 (proximally diminishing radius and distally increasing radius) over which sleeve 160 is distally passed to compress jaws 55 to engage a cooperating implant.

FIG. 8 shows a system of the present invention. FIG. 8 shows an implant 100. Implant 100 has a sidewall 520 and endwalls or top and bottom walls or surfaces 530, 540. When implanted, endwalls 530, 540 in FIG. 8 become one or the top surface or bottom surface. Openings in the sidewall or wall 520 of implant 100 define cooperating surfaces 500 which are engaged by prongs or jaws 55, when sleeve 160 is distally urged. An optional locator pin 84 cooperates with and engages an optional sidewall hole 510 also defined by the sidewall 520 of implant 100.

While the invention has been illustrated and described in detail in the drawings and foregoing description, the same is to be considered as illustrative and not restrictive in character, it being understood that only the preferred embodiments have been shown and described and that all changes and modifications that come within the spirit of the invention are desired to be protected.

What is claimed is as follows:

1. An insertion device for holding, maneuvering and deploying an intervertebral implant, comprising:
a handle;
a shaft having a proximal end and a distal end, said proximal end attached to said handle;
a pair of jaws attached to the shaft, each of said jaw pair having a distal free end, each of said free ends defining a gripping element, said free ends of said jaws movable between gripping positions to grasp the implant between said gripping elements and releasing positions to release the implant;
a hollow sleeve slideably mounted on said shaft, said hollow sleeve having a proximal end and an opposite distal end, said hollow sleeve defining an inner bore for receiving said shaft and said first ends of said jaws, said sleeve slidable between a first position adjacent said handle and a second engaging position, said sleeve forcing said jaws together when said sleeve is in the engaging position,
biasing means mounted on said shaft, said biasing means distally urging said hollow sleeve so as to urge said jaws toward their engaging position.

2. A device of claim 1 wherein the distal end of the hollow sleeve defines a plurality of partially circumferential steps, the steps having increasing proximal circumferential radii.

3. A device of claim 1 wherein there is a locator pin projecting distally from said shaft.

4. A device of claim 1 which further includes a collar and a biasing means, the collar being coaxially located of the shaft between the handle and the hollow sleeve, the biasing means being coaxially located on the shaft between the collar and the proximal end of the hollow sleeve and being seated within the collar.

5. A device of claim 4 wherein the collar is integrally formed with the hollow sleeve on its proximal end and is a part thereof.

6. A device of claim 4 wherein the biasing means is a spring.

7. A device of claim 1 wherein the shaft has an external thread and the collar has a cooperating internal thread, the collar thereby being threadingly engaged to the shaft.

8. A device of claim 5 which further includes a lock nut coaxially located on the shaft between the handle and the proximal side of the collar, the shaft having an external thread, the lock nut having a cooperating internal thread such that the lock nut can restrict proximal movement of the collar.

9. A medical device system comprising an insertion device of claim 1 and at least one intervertebral implant with a sidewall which defines surfaces to cooperate with the jaws to be held by the insertion device.

10. A system according to claim 9 wherein the system is a kit including an inserter and a plurality of implants of different sizes.
